(19) United States
(12) Patent Application Publication
(10) Pub. No.: US 2020/0397804 A1
(43) Pub. Date: Dec. 24, 2020

(54) TREATMENT AND PREVENTION OF NEURODEGENERATIVE DISORDERS

(71) Applicant: Martha M. Hellstrom, (US)

(72) Inventor: Harold Richard Hellstrom, Fayetteville, NY (US)

(21) Appl. No.: 16/907,210

(22) Filed: Jun. 20, 2020

Related U.S. Application Data
(60) Provisional application No. 62/864,406, filed on Jun. 20, 2019.

(51) Int. Cl. A61K 31/683; A61K 31/404; A61K 31/353; A61K 31/138; A61K 31/4025; A61K 31/27; A61K 31/661; A61K 9/00

(52) U.S. Cl. CPC A61K 31/683; A61K 31/404; A61K 31/353; A61K 31/138; A61K 9/0048; A61K 31/27; A61K 31/661; A61K 9/0019; A61K 31/4025

(57) ABSTRACT

Provided are methods and compositions for treatment and/or prevention of neurodegenerative disorders which may use parasympathomimetic agents and/or anti-sympathetic agents.

### TECHNICAL FIELD

Provided are methods and compositions for treatment and/or prevention of neurodegenerative disorders by ophthalmic administration of one or more agents, which may use parasympathomimetic agents and/or anti-sympathetic agents.

### BACKGROUND

The incidence of age-related disorders sharply increases with advanced age. The prevalence of dementia is below 1% in individuals 60-64 but is between 24% and 33% for those 85 or older (Blennow K, de Leon M J, Zetterberg H. Alzheimer's disease. Lancet 2006; 368:387-403). There is also a 10 fold inrease in the risk of fibrillation from about middle age to aged (Go et al. (2001) “Prevalence of diagnosed atrial fibrillation in adults: national implications for rhythm management and stroke prevention: The Anticoagulation and Risk Factors in Atrial Fibrillation (ATRIA) Study”, JAMA 285:2370-5) and the risk of garden variety stroke rises rapidly by more than a factor of 10 in the very aged (Mozaffarian et al. (2015) “Heart disease and stroke statistics—2015 update: a report from the American Heart Association”, Circulation 131:e29-322).

### Neurodegenerative Disorders

One type of age-related disorders is a neurodegenerative disorder. This type of disorder is a disabling disorder whose subtypes are characterized by damage and death of neurons. However, a neurodegenerative disorder may also occur at any age. As will be set forth below, there are intraocular neurodegenerative disorders, such as glaucoma, age-related macular degeneration (AMD), diabetic retinopathy, retinitis pigmentosa, retinitis of prematurity, and retinal vein occlusion, and cerebral neurodegenerative disorder, such as Alzheimer's disease, Parkinson's disease, Huntington's disease, amyotrophic lateral sclerosis, and Lewy body disease (or dementia).

Five Pathologies have been found to be associated with neurodegenerative disorders. The five pathologies include increased levels of glutamate, inflammation, ischemic injury/vasoconstriction, and amyloid, and decreased levels of acetylcholine. These five pathologies are considered to represent dysregulation of neuronal autoregulation. The components of three pathologies seem especially concerned with basic neuronal autoregulation: Controlling neurotransmission (acetylcholine), controlling neuronal activation (glutamate), and assuring proper blood flow with different levels of neuronal metabolism (neuronal vascular supply). In particular, increased levels of glutamate have been found to be associated with AMD (Liberatore et al. (2017) “Permissive role for mGlu1 metabotropic glutamate receptors in excitotoxic retinal degeneration”, Neuroscience 363:142-9); glaucoma (Harada et al. (2007) “The potential role of glutamate transporters in the pathogenesis of normal tension glaucoma”, J Clin Invest 117:1763-70 and Liberatore et al. (2017) “Permissive role for mGlu1 metabotropic glutamate receptors in excitotoxic retinal degeneration”, Neuroscience 363:142-9); diabetic neuropathy (Coughlin et al. (2017) “Muller cells and diabetic retinopathy”, Vision Res 139:93-100), retinal vein occlusion (Wakabayashi et al. (2006) “Glutamate levels in aqueous humor of patients with retinal artery occlusion”, Retina 26:432-6 and Mosinger et al. (1991) “Blockade of both NMDA and non-NMDA receptors is required for optimal protection against ischemic neuronal degeneration in the in vivo adult mammalian retina”, Exp Neurol 113:10-7) retinitis pigmentosa (Mosinger et al. (1991) “Blockade of both NMDA and non-NMDA receptors is required for optimal protection against ischemic neuronal degeneration in the in vivo adult mammalian retina”, Exp Neurol 113:10-7), retinopathy of prematurity (Mosinger et al. (1991) “Blockade of both NMDA and non-NMDA receptors is required for optimal protection against ischemic neuronal degeneration in the in vivo adult mammalian retina”, Exp Neurol 113:10-7), Alzheimer' s disease (Wakabayashi et al. (2006) “Glutamate levels in aqueous humor of patients with retinal artery occlusion”, Retina 26:432-6 and Lewerenz et al. (2015) “Chronic glutamate toxicity in neurodegenerative diseases—what is the evidence?” Front Neurosci 9:469); Parkinson' s disease (Johnson et al. (2009) “Glutamate receptors as therapeutic targets for Parkinson's disease. CNS”, Neurol Disord Drug Targets 8:475-91), Huntington's disease (Lewerenz et al. (2015) “Chronic glutamate toxicity in neurodegenerative diseases—what is the evidence?” Front Neurosci 9:469) amyotrophic lateral sclerosis (Lewerenz et al. (2015) “Chronic glutamate toxicity in neurodegenerative diseases—what is the evidence?” Front Neurosci 9:469 and Le Verche et al. (2011) “Glutamate pathway implication in amyotrophic lateral sclerosis: what is the signal in the noise”, J Receptor Ligand Channel Res 4:1-22) and Lewy body dementia (Price et al. (2010) “Alterations in mGluR5 expression and signaling in Lewy Body Disease and in transgenic models of alpha-synucleinopathy—implications for excitotoxicity”, PLoS ONE 5:e14020). Increased levels of inflammation has been found to be associated with AMD (Knickelbein et al. (2015) “Inflammatory Mechanisms of Age-related Macular Degeneration”, Int Ophthalmol Clin 55:63-78), glaucoma (Bukhari et al. (2016) “Microvascular endothelial function and sevrity of open angle glaucoma”, Eye 30:1579-87 and Soto H et al. (2014) “The complex role of neuroinflammation in glaucoma”, Cold Spring Harb Perspect Med 4:a017269), diabetic neuropathy (Moran et al. (2016) “Neurovascular cross talk in diabetic retinopathy: Pathophysiological roles and therapeutic implications”, Am J Physiol Heart Circ Physiol 311:H738-H749 and Whitcup et al. (2013) “Inflammation in retinal disease”, Int J Infl Article ID 724648, 4, pages), retinal vein occlusion (Whitcup et al. (2013) “Inflammation in retinal disease”, Int J Infl Article ID 724648, 4 pages and Deobhakta et al. (2013) “Inflammation in retinal vein occlusion”, Int J Inflam Article ID 438412, 6 pages), retinitis pigmentosa (McMurtrey et al. (2018) “A review of the immunologic findings observed in retinitis pigmentosa”, Surv Ophthalmol 63:769-81) and retinopathy of prematurity (Wang et al. (2015) “Pigment epithelium-derived factor regulates glutamine synthetase and l-glutamate/l-aspartate transporter in retinas with oxygen-induced retinopathy”, Curr Eye Res 40:1232-44 and Weinberger et al. (2002) “Oxygen toxicity in premature infants”, Toxicol Appl Pharmacol 181:60-7), Alzheimer's disease (Leonard (2004) “Pharmacotherapy in the treatment of Alzheimer's disease: An update”, World Psychiart 3:84-8 and Cacabelos (2007) “Donepezil in Alzheimer's disease: From conventional trials to pharmacogenetics”. Neuropsychiatr Dis Treat 3:303-33), Parkinson's disease (Gelders et al. (2018) “Linking neuroinflammation and neurodegeneration in Parkinson's disease”, J Immunol Res 2018: Article ID 4784268, 12 pages) Huntington's disease (Rocha et al. (2016) “Neuroimmunology of Huntington's disease: Revisiting evidence from human studies”, Mediators Inflamm 2016:Article ID 8653132, 10 pages), amyotrophic lateral sclerosis (Liu et al. (2017) “Role of neuroinflammation in amyotrophic lateral sclerosis: Cellular mechanisms and therapeutic implications”, Front Med Neurosci 8:1005) and Lewy body dementia (Surendranatan et al. (2018) “Early microglial activation and peripheral inflammation in dementia with Lewy bodies”, Brain 141:3418-27). Increased levels of ischemic injury/vasoconstriction have been found to be associated with AMD (Coleman et al. (2013) “Age-related macular degeneration: choroidal ischaemia?” Br J Ophthal 97:1020-3), glaucoma (Stefansson et al. (2017) “Retinal Oximetry Discovers Novel Biomarkers in Retinal and Brain Diseases”, Invest Ophthalmol Vis Sc; 58:BIO227-BIO233 and Luo et al. (2015), “Ocular blood flow autoregulation mechanisms and methods”, J Ophthalmol 2015: Article IE 864871, 7 pages), diabetic retinopathy (Moran et al. (2016) “Neurovascular cross talk in diabetic retinopathy: Pathophysiological roles and therapeutic implications”., Am J Physiol Heart Circ Physiol 311:H738-H749 and Stefansson et al. (2017) “Retinal Oximetry Discovers Novel Biomarkers in Retinal and Brain Diseases”, Invest Ophthalmol Vis Sc; 58:BIO227-BIO233), retinal vein occlusion (Stefansson et al. (2017) “Retinal Oximetry Discovers Novel Biomarkers in Retinal and Brain Diseases”, Invest Ophthalmol Vis Sc; 58:BIO227-BIO233 and Wong et al. (2010) “Clinical practice. Retinal-vein occlusion”, N Engl J Med 363:2135-44), retinitis pigmentosa (Stefansson et al. (2017) “Retinal Oximetry Discovers Novel Biomarkers in Retinal and Brain Diseases”, Invest Ophthalmol Vis Sc; 58:BIO227-BIO233), retinopathy of prematurity (Weinberger et al. (2002) “Oxygen toxicity in premature infants”, Toxicol Appl Pharmacol 181:60-7) and Alzheimer's Disease (Blennow et al. (2006) “Alzheimer's disease”, Lancet 368:387-403 and Claassen et al. (2006), “Cholinergically mediated augmentation of cerebral perfusion in Alzheimer's disease and related cognitive disorders: The cholinergic-vascular hypothesis”, J Gerentol Med Sci 61A:267-71). Accumulation of amyloid protein have been found to be associated with AMD (Gupta et al. (2016) “One protein, multiple pathologies: multifaceted involvement of amyloid beta in neurodegenerative disorders of the brain and retina. [Review]”, Cell Mol Life Sci 73:4279-97), glaucoma (Gupta et al. (2016) “One protein, multiple pathologies: multifaceted involvement of amyloid beta in neurodegenerative disorders of the brain and retina. [Review]”, Cell Mol Life Sci 73:4279-97), Alzheimer's disease (Blennow et al. (2006) “Alzheimer's disease”, Lancet 368:387-403), Parkinson's disease (Petrou et al. (2015) “Amyloid deposition in Parkinson disease and cognitive impairment: A systematic Review”. Mov Discord 30:928-35), Huntington's disease (Le Verche et al. (2011) “Glutamate pathway implication in amyotrophic lateral sclerosis: what is the signal in the noise”, J Receptor Ligand Channel Res 4:1-22), amyotrophic lateral sclerosis (Joon et al. (2009) “Intracellular amyloid beta interacts with SODl and impairs the enzymatic activity of SODl: Implications for the pathogenesis of amyomyotrophic lateral sclerosis”, Exp Mol Med 41:611-7) and Lewy body dementia (Joon et al. (2009) “Intracellular amyloid beta interacts with SOD1 and impairs the enzymatic activity of SOD1: Implications for the pathogenesis of amyomyotrophic lateral sclerosis”, Exp Mol Med 41:611-7). Acetylcholine has been found to be a neurotransmitter in the vertebrate retina (Hutchins (1987) “Acetylcholine as a neurotransmitter in the vertebrate retina”, Exp Eye Res; 45:1-38). Furthermore, decreased acetylcholine, or disturbances of acetylcholine, is recognized with Alzheimer's disease (Cacabelos (2007) “Donepezil in Alzheimer's disease: From conventional trials to pharmacogenetics”. Neuropsychiatr Dis Treat 3:303-33 and Blennow et al. (2006) “Alzheimer's disease”, Lancet 368:387-403), Parkinson's disease (Bohnen et al., (2011) “The cholinergic system and Parkinson Disease”, Behav Brain Res 221:564-73), Huntington's disease (Smith et al. (2006) “Cholinergic neuronal defect withot cell loss in Huntington's disease”, Hum Mol Gen 15:3119-31), amyotrophic lateral sclerosis (Campanari et al. (2016) “Neuromuscular junction impairment in amyotrophic lateral sclerosis: Reassessing the role of acetylcholine”, Front Med Neurosci 9:160) and Lewy body dementia (Kitajima et al. (2015) “A review of the role of anticholinergic activity in Lewy body disease and delirium”, Neurodegener Dis 15:162-7).

Attempts have been made to treat these disorders (see, for example (Cacabelos (2007) “Donepezil in Alzheimer's disease: From conventional trials to pharmacogenetics”. Neuropsychiatr Dis Treat 3:303-33, Rocha et al. (2016) “Neuroimmunology of Huntington's disease: Revisiting evidence from human studies”, Mediators Inflamm 2016:Article ID 8653132, 10 pages, Pfeiffer et al. (2013) “Neuroprotection of medical IOP-lowering therapy”, Cell Tissue Res 353:245-51, Chang et al. (2015), “Angiotensin II in inflammation, immunity and rheumatoid arthritis”, Clin Exp Immunol 179:137-45, Nation et al. (2016) “Alzheimer's Disease Neuroimaging Initiative. Older Adults Taking AT1-Receptor Blockers Exhibit Reduced Cerebral Amyloid Retention”, J Alzheimer's 50:779-89, Hajjar et al. (2013) “Do angiotensin receptor blockers prevent Alzheimer's disease?” Curr Opin Cardiol 28:417-25 and Kim et al. (2017) “Beyond symptomatic effects: potential of donepezil as a neuroprotective agent and disease modifier in Alzheimer's disease”, Br J Pharmacol 174:4224-32). Angiotensin Receptor Blockers have been suggested to have a neuroprotective effect. none of these treatments are curative and some have serious side effects (Craig et al. TFOS DEWS II Report Executive Summary. Ocul Surt 2017; http://dx.doi.org/10.1016/j.jtos2017.08.003).

### Homeostatasis Shift

The 2007 edition of the altered homeostatic theory is the latest reported presentation of this theory (Hellstrom (2007) “The altered homeostatic theory: A hypothesis proposed to be useful in understanding and preventing ischemic heart disease, hypertension, and diabetes—including reducing the risk of age and atherosclerosis” Med Hypotheses 68:415-473). Later, other age-related disorders were added, including neurodegenerative disorders.

The altered homeostatic theory argues that risk factors, usually identified with risk factors for ischemic heart disease (cardiovascular risk factors), favor multiple age-related disorders by inducing chronic sympathetic activation. The theory also asserts that preventative factors improve age-related disorders by healing preventative factor-induced parasympathetic activation. Tables 1 and 2 in the 2007 Hellstrom paper (referenced above) provide evidence to support the relation of risk factors to sympathetic activation and disease, and preventative factors to parasympathetic activation and health. Additionally, risk factors for Alzheimer's disease, dry eye disease (DED) and age-related macular degeneration have been described as “cardiovascular risk factors” (Baumgart et al (2015) “Summary of the evidence on modifiable risk factors for cognitive decline and dementia”, Alzheimers Dement 11:718-726 and Fraser-Bell et al (2008) “Cardiovascular risk factors and age-related macular degeneration: The Los Angeles Latino Eye Study”, Am J Ophthalmol 145(2); 308-316). A basic premise

A basic premise of the altered homeostatic theory is that helpful acute bodily changes of defensive fight/flight, when prolonged chronically, cause disease. As example, to provide the needed added energy for the exertions of acute fight/flight, the blood stream is flooded with lipids and glucose. With chronic sympathetic activation, this results in dyslipidemia (IHD) and insulin resistance (diabetes). Of interest, dyslipidemia and inflammation have also been related to ND (Hallett et al. (2019) “Lipid and immune abnormalities causing age-dependent neurodegeneration and Parkinson's disease”, J Neuroinflamm 16:153).

### SUMMARY

Provided is a method for preventing and/or treating one or more neurodegenerative disorders comprising ophthalmically administering to a subject in need thereof one or more agents in an amount effective to (a) shift homeostasis toward parasympathetic dominance; (b) modulate activities of neurons by the autonomic nervous system; (c) normalize neuronal autoregulation or (d) any combination thereof. Specifically, said method may be used to (a) shift homeostasis toward parasympathetic dominance and/or (b) modulate activities of neurons by the autonomic nervous system; and/or (c) normalize neuronal autoregulation. Said agent may be administered topically, or via intraocular or intravitreal injection. Said subject may be a human or other mammal.

As set forth above, a neurodegenerative disorder is a disorder whose subtypes are characterized by damage and death of neurons. In one embodiment, the neurodegenerative disorder is an intraocular disorder, and may include but is not limited to glaucoma, AMD, diabetic retinopathy, retinitis pigmentosa, retinal vein occlusion, and retinopathy of prematurity. In another embodiment, the neurodegenerative disorder is a cerebral neurodegenerative disorder which may include but is not limited to Parkinson's disease, Alzheimer's disease, Lewy body dementia, or amyotrophic lateral sclerosis, and Huntington's disease. In a particular embodiment, the neurodegenerative disorder may result from neuronal autoregulation dysfunction.

In yet another particular embodiment, said agents may be parasympathomimetic agents and/or antisympathetic agents which may be administered singly or in combination. In one embodiment, at least one anti-sympathetic agent is administered to said subject. In another embodiment, at least one parasympathomimetic agent is administered to said subject. In yet another embodiment, at least one anti-sympathetic agent and at least one parasympathomimetic agent is administered to said subject. In a particular embodiment, the anti-sympathetic agent and parasympathomimetic agent is administered in the form of a composition.

In a particular embodiment said agent(s) is administered at least about 5% lower than its lowest effective therapeutic ophthalmic dose. In a more particular embodiment said agent(s) is administered between about 5% to about 10% below its known effective therapeutic ophthalmic dose. In an even more particular embodiment, said agent is administered at a dose of at least about 98% to about 5% below its known effective therapeutic ophthalmic dose.

In a particular embodiment, the agent(s) used in the methods set forth above may be administered to at risk individuals, e.g., those 60 years old and above or with a family history of a particular disorder.

### DEFINITIONS

Where a range of values are provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

All publications and patents cited in this disclosure are incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

It must be noted that as used herein and in the appended claims, the singular forms “a,” “and” and “the” include plural references unless the context clearly dictates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the present invention. Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. Thus, the terms “comprising”, “including,” containing”, “having” etc. shall be read expansively or open-ended and without limitation. When used herein, the term “comprising” can be substituted with the term “containing” or sometimes when used herein with the term “having”.

The phrases “effective amount” or “amount effective” are art-recognized terms, and refer to an amount of an agent that, when incorporated into compositions set forth herein, produces some desired effect at a reasonable benefit/risk ratio applicable to any medical treatment. In certain embodiments, the term refers to that amount necessary or sufficient to eliminate, reduce or maintain (e.g., prevent the spread of) a symptom of a neurodegenerative disorder. The effective amount may vary depending on such factors as the disease or condition being treated, the particular composition being administered, or the severity of the disease or condition. One of skill in the art may empirically determine the effective amount of a particular agent without necessitating undue experimentation.

As defined herein, an “agent” is a chemical substance that affects the functioning of living things.

The terms “composition(s)” and “formulation(s)” may be used interchangeably.

As defined herein, a “physiological parasympathetic homeostatic shift” means to shift homeostasis toward parasympathetic dominance where there is an increase in parasympathetic activity, which in a specific embodiment, results from parasympathetic activation and a corresponding decrease in sympathetic activity.

As defined herein, the terms “treat”, “treatment” and “treating” are to be understood accordingly as embracing prophylaxis and treatment or amelioration of symptoms of disease as well as treatment of the cause of the disease.

As defined herein, “neuronal autoregulation” includes but is not limited to controlling levels of glutamate, inflammation, blood flow of vessels suppling neurons, acetylcholine, amyloid and ocular flow autoregulation.

As defined herein, “ocular flow autoregulation” is the ability to maintain a relatively constant level of blood flow in the presence of changes to ocular perfusion pressure and varied metabolic demand.

### DETAILED DESCRIPTION OF PREFERRED EMBODIMENTS

As noted above, provided is a method for preventing and/or treating one or more neurodegenerative disorders comprising topically administering to a subject in need thereof one or more agents in an amount effective to shift homeostasis toward parasympathetic dominance and particularly parasympathetic activation. Said agents may be administered to those subjects afflicted with the disorder or those subjects at risk for said disorder (e.g., individuals having one or more of the risk factors for a particular disorder). Parasympathetic homeostatic shift and in a specific embodiment, by parasympathetic activation may be determined by measuring the level of chronic sympathetic activation in said subject, such as electrophotonic imaging (see, for example, Deo (2015) “Effect of Anapanasati meditation technique through electrophotonic imaging parameters: A pilot study”, International Journal of Yoga 8(2):117-21 and Sukhsohale et al. (2012) “Effect of short-term and long-term Brahmakumaris Raja Yoga meditation on physiological variables”. Indian J Physiol Pharmacol 56:388-92) as well as other brain imaging techniques known in the art. This method may be used to measure the effectiveness of said agents in preventing neurodegenerative disorders by using such techniques on at risk subjects and monitoring results of said agents on sympathetic activation at various time periods after administration of said agents. It may also be used to monitor the effectiveness of treatment of afflicted subjects.

Said agents may, also, modulate activities of neurons by the autonomic nervous system. The autonomic nervous system contains a dynamic tension between sympathetic and parasympathetic activation. Importantly, the autonomic nervous system autoregulates multiple intraocular and body functions (McDougal and Gamlin (2015) “Autonomic control of the eye”, Compr Physiol 5:439-73 and Beissner et al (2013) “The autonomic brain: An activation likelihood estimation meta-analysis for central processing of autonomic function”, J Neurosci 33:10503-11).

As the autonomic nervous system autoregulates, a risk factor-induced chronic sympathetic activation directly and adversely affects the normal autonomic nervous system's autoregulatory balance between sympathetic and parasympathetic activation—prompting an unhealthy intraocular (and cerebral) “sympathetic” state. Oppositely, “parasympathetic” eye drops used in the method set forth above prompt a healing intraocular “parasympathetic” state—and have some positive effect for the cerebrum.

Autonomic nervous system activity may be detected using methods known in the art, such as imaging (see, for example, Beissner et al. (2013), “The autonomic brain: An activation likelihood estimation meta-analysis for central processing of autonomic function”, J Neurosci 33:10503-11).

In a particular embodiment, the neurodegenerative disorder results from neuronal autoregulation dysfunction. Thus, provided is a method for preventing and/or treating one or more neurodegenerative disorders resulting from neuronal autoregulation dysfunction in a subject in need thereof comprising topically administering an amount of one or more agents in an effective to normalize neuronal autoregulation.

Neuronal autoregulation includes but is not limited to ocular flow autoregulation and normalizing levels of glutamate, inflammation, in the eye and/or brain; amyloid, vasodilation activity and acetylcholine level in a subject.

The degree of neuronal activity may be determined using methods known in the art. Said measurements may be used to monitor the effectiveness of agents in preventing or treating a neurodegenerative disorder. Glutamate levels may be determined by direct measurement (see, for example, Wakabayashi et al. (2006) “Glutamate levels in aqueous humor of patients with retinal artery occlusion”, Retina 26:432-6) or by measuring glutamate receptor activity (see, for example, Lewerenz et al. (2015) “Chronic glutamate toxicity in neurodegenerative diseases—what is the evidence?” Front Neurosci 9:469). Inflammation in the eye may be measured, for example by measuring the level of inflammatory mediators such as prostaglandin E2, tumor necrosis factor, nitric oxide, interleukin-8 (IL-8), IL-9, IL-10, IL-12, interferon alpha (IFN-α), interferon gamma IFN-γ in an aqueous sample or alternatively by (Chua et al. (2012) “Expression profile of inflammatory cytokines in aqueous from glaucomatous eyes”, Mol Vis. 18:431-8), slit lamp technique (Askoy et al. (2015) “Evaluation of anterior chamber inflammation”, Indian J Ophthalmol. 63: 288-289) or by laser flare/cell meter (see Gupta et al. (2013) “Ancillary investigations in uveitis”. Indian J Ophthalmol. 61:263-8). Inflammation in the brain may be detected and/or quantitated by measuring cytokine levels in blood or cerebrospinal samples from subjects (e.g., IL1β, IL2, IL6, IL-34, IFNγ, TNF receptor 1 and TNFα) or by MRI or PET imaging (see, for example Gelders et al. (2018)“Linking neuroinflammation and neurodegeneration in Parkinson's disease”, J Immunol Res 2018:Article ID 4784268, 12 pages and Surendranatan, et al. (2018) “Early microglial activation and peripheral inflammation in dementia with Lewy bodies”, Brain 141:3418-27). Vasodilation may be measured by for example, by flow-mediated vasodilation (see, for example Khurana et al. (2004) “Vasodilatory effects of cholinergic agonists are greatly diminished in aorta from M3R−/− mice”, Eur J Pharmacol 493:127-32). Beta-amyloid levels may be measured in cerebrospinal fluid of subjects (see, for example, Toledo et al. (2012) “CSF biomarkers cutoffs: the importance of coincident neuropathological diseases”. Acta Neuropathol. 124:23-35) or PET scans (Clark et al. (2011) “AV45-A07 Study Group. Use of florbetapir-PET for imaging beta-amyloid pathology”, JAMA 305:275-283). Acetylcholine levels may be measured using commercially available colorimetric assays.

In a particular aspect, as set forth above, neurodegenerative disorders such as Alzheimer's Disease and AMD have been attributed to risk factor-induced harmful sympathetic activation. In another particular aspect, chronic sympathetic activation favors neuronal autoregulatory dysregulation and thus favoring the five pathologies, increased levels of glutamate, inflammation, ischemic injury/vasoconstriction, and amyloid, and decreased levels acetylcholine. The agents used in the method set forth above shift the intraocular milieu from harmful sympathetic activation to healing parasympathetic dominance and improve neuronal autoregulation and heal the five pathologies. Thus, said agents may in addition to normalizing neuronal autoregulation, may also shift homeostasis toward parasympathetic dominance. Furthermore, given that in one embodiment, said agents may in addition to shifting homeostasis toward parasympathetic dominance, modulate activities of neurons by the autonomic nervous system, said agents may also act to normalize neuronal autoregulation, shift homeostasis toward parasympathetic dominance and modulate activities of neurons by the autonomic nervous system.

As set forth above, said neurodegenerative disorder may be an intraocular disorder or cerebral neurodegenerative disorder. Treatment of cerebral neurodegenerative disorders may in a particular embodiment be due to the occurrence of eye/brain neurotransmission. In a particular embodiment, for treating cerebral ND, the intraocular “parasympathetic” status from eye drops is transferred to the brain by eye/brain neurotransmission, where it favors improving cerebral ND.

The ophthalmic agents used in the method set forth above, after traversing the scleral barrier, make the interior of the eye “healthy.” This “healthy” intraocular status is then transferred to the brain by eye/brain neurotransmission, where it favors improving cerebral pathologies—and thus improving cerebral neurodegenerative disorders. In a particular embodiment, eye/brain neurotransmission is based on the cholinergic system., since the eye has cholinergic neurons (Yasuhara et al. (2003) “Demonstration of cholinergic ganglion cells in rat retina: expression of an alternative splice variant of choline acetyltransferase”, Journal of Neurosci 23:2872-81). The cholinergic system is based on acetylcholine (Yasuhara et al. (2003) “Demonstration of cholinergic ganglion cells in rat retina: expression of an alternative splice variant of choline acetyltransferase”, Journal of Neurosci 23:2872-81; Gieslow et al. (2017) “The input-output relationship of the cholinertgic basal forebrain”, Cell Rep 18:1717-830” and Erskine et al. (2014) “Connecting the retina to the brain”, ASN News DOI 10.1177/1759091414562107:1-28. and acetylcholine acts as a neurotransmitter in the retina (Yasuhara et al. (2003) “Demonstration of cholinergic ganglion cells in rat retina: expression of an alternative splice variant of choline acetyltransferase”, Journal of Neurosci 23:2872-81). The cholinergic system is diffusely spread throughout the brain (Gieslow et al. (2017) The input-output relationship of the cholinertgic basal forebrain. Cell Rep 18:1717-830.

### Parasympathomimetic Agents

There are multiple parasympathomimetic agents, and several are formulated for topical use. These include but are not limited to pilocarpine, carbachol, ecothiopate, demecarium bromide and diisopropyl fluorophosphate mentioned. In a specific embodiment, the parasympathomimetic agent may be carbachol, echothiophate iodide, physostigmine and/or demecarium bromide. More than one parasympathomimetic agent may be used to secure a wider spectrum of results.

### Sympathetic Blockers/Anti-Sympathetic Agents

Sympathetic blockers are either alpha or beta selective. They may also act as alpha and beta-blockers (see, for example carvedilol and labetalol).

In another specific embodiment, the sympathetic blocker or anti-sympathetic agent may be a beta-blocker. The beta-blocker may be a beta-selective or non-selective agent and may include but is not limited to atenolol (selective), bisoprolol (selective), nevbiol (selective), propanolol (nonselective) timolol (nonselective), betaxolol (beta 1 selective antagonist), levobunolol (nonselective beta 1 and 2 blocking agent), carteolol (nonselective beta-blocker), metipranolol (nonselective beta-blocker) levobetaxolol (beta 1 inhibitor) as well as nitric oxide (NO) donors such as nipradilol (nonselectable beta-blocker), carvediol (non-selective) and nebivolol (betal selective blocker) and additionally, substances having or intrinsic sympathetic action (ISA) such as oxyprenolol and pindolol.

### Angiotensin Blockers

Also provided are angiotensin blockers which include, but are not limited to, ACE inhibitors and angiotensin II receptor blockers (ARBs). Angiotensin blockers may, as noted above, may act as anti-sympathetic agents as well as parasympathomimetic agents. Examples of ACE inhibitors include but are not limited to benazepril, captopril, enapapril, fosinopril, lisinopril, moexipril, perindopril, quinapril, ramipril and trandolapril. Examples of angiotensin II receptor antagonists include but are not limited to Losartan, Valsartan, Irbesartan, Candesartan, Telmisartan, Eprosartan, Olmesartan, Azilsartan.

### Dosages

As noted above, in one embodiment, at least one of the agents set forth above is administered at a lower dose than of its lowest known effective therapeutic ophthalmic dose, the dose administered to the eye. High doses, as used for glaucoma, cause harmful tissue injury (Zhang Y et al. “Influence of pilocarpine and timolol on human meibomian gland epithelial cells”, Cornea 36:719-724)). Drug-induced injury could complicate the inherent neuronal injury of neurodegenerative disorders, and injury also causes harmful sympathetic activation (Barton RN. (1987) “The neuroendocrinology of physical injury”, Baillieres Clin Endocrinol Metab 1:355-74 and Kidd B et a; (1992) “Role of the sympathetic nervous system in chronic joint pain and inflammation”, Ann Rheum Dis 1992;51:1188-91).

Therefore, in a particular embodiment, doses used in the method set forth above should be rather dilute to avoid drug-induced toxicity, because the neurodegenerative process itself causes neuronal injury, and because injury causes harmful sympathetic activation.

In a particular embodiment, to help ensure that initial doses are non toxic, the strength of initial topical testing doses of at least one agent should be about 10% of the lowest effective therapeutic ophthalmicdose. Information about lowest effective therapeutic ophthalmic doses for said agents could be obtained from various Opthalmic treatment resources (e.g, Manual of Ocular Diagnosis and Therapy 6e and Willis Eye Manual: Office and Emergency Room Diagnosis and Treatment of Eye Diseases 5e). From initial testing doses, subsequent doses may be adjusted up or down by about 50% to 100% to determine maximally effective therapeutic doses. If there is doubt that a particular agent is especially liable to toxicity, the dose of 10% of the lowest therapeutic level can be tested for toxicity by the method of of Zhang (Zhang et al (2017) “Influence of Pilocarpine and Timolol on Human Meibomian Gland Epithelial Cells”, Cornea 36:719-24).

In another embodiment, the lowest effective therapeutic ophthalmic dose of a particular agent may be determined by comparing therapeutic oral dose levels of a particular agent to therapeutic oral doses from parasympathetic agents which have been used topically—as pilocarpine. If the therapeutic oral dose of the tested agent is similar to the therapeutic oral dose of pilocarpine, then the initial topical dose of the tested drug would be 10% of 1% (the lowest topical dose of pilocarpine for glaucoma (Wu LL and Huang P (2011) “A 12-week, double-masked, parallel-group study of the safety and efficacy of travoprost 0.004% compared with pilocarpine 1% in Chinese patients with primary angle-closure and primary anagle-closure glaucoma”, J Glaucoma 388-91)). If not, doses could be modified accordingly. It is noted that oral pilocarpine, at an oral dose of 5 mg, was used to treat Sjogren's syndrome (American Academy of Ophthalmology Cornea/External Disease Panel. Preferred Practice Pattern® Guidelines. Dry Eye Syndrome. San Francisco Calif. American Academy of ophthalmology; 2013. Available at www.aao.org/ppp).

In a particular embodiment, at least one of the agents is administered at a dose at least about 5% lower or 10% lower than its lowest effective therapeutic ophthalmic dose. The object is to administer effective dosages but limit neuronal injury. In an even more particular embodiment, at least one of said agents are administered at a dose of at least about 98% to 5% below its lowest effective therapeutic topical dose. In even more particular embodiments, the subject is administered at least one of said agents at a dose at least about 95%, 90%, 85%, 80%, 75%, 70%, 65%, 60%, 55%, 50%, 45%, 40%, 35%, 30%, 25%, 20% or 15% below its lowest effective therapeutic ophthalmic dose. In a specific embodiment, all of agents used would be at least about 5% lower than that its lowest effective therapeutic ophthalmic dose. In an even more particular embodiment, at least one of the agents set forth above is administered at a lower dose than of any known therapeutic dose level for glaucoma. In a particular embodiment, at least one of the agents is administered at a dose at least about 5% lower or 10% lower than that used to treat glaucoma. In an even more particular embodiment, at least one of said agents are administered at a dose of at least about 98% to 5% below the known effective treatment of glaucoma. In even more particular embodiments, the subject is administered at least one of said agents at a dose at least about 95%, 90%, 85%, 80%, 75%, 70%, 65%, 60%, 55%, 50%, 45%, 40%, 35%, 30%, 25%, 20% or 15% below the known effective treatment of glaucoma. In a specific embodiment, all of agents used would be at least about 5% lower than that used to treat glaucoma.

The agents set forth above may be administered once, twice or three times a day. In one embodiment, the two agents would be administered in one formulation or together. In a particular embodiment, the two agents are administered in one formulation.

In a specific embodiment, a parasympathomimetic agent, may be administered at around 0.01%, to about 0.5% w/v or around 0.05% to about 0.1% w/v or about 0.075% to about 0.2% w/v and an anti-sympathetic agent, such as a beta-blocker with nitroglycerin-like vasodilative activities may be administered at a dose of about 0.01% to about 0.1% w/v. or between about 0.01% to about 0.05% w/v.

### Compositions/Formulations

In certain embodiments, the composition comprises at least one parasympathomimetic and at least one anti-sympathetic agent. Preferably, the compositions will be formulated as solutions, suspensions and other dosage forms for ophthalmic administration in a pharmaceutically acceptable carrier, adjuvant, or vehicle. Aqueous solutions are generally preferred, based on ease of formulation, as well as a patient's ability to easily administer such compositions by means of instilling one to two drops of the solutions in the affected eyes. However, the compositions may also be suspensions, viscous or semi-viscous gels, ointments or other types of solid or semi-solid compositions.

Any of a variety of carriers may be used in the ophthalmic formulations including water, mixtures of water and water-miscible solvents, such as C1- to C7-alkanols, vegetable oils or mineral oils comprising from 0.5 to 5% non-toxic water-soluble polymers, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carpool, or mixtures of those polymers. The concentration of the carrier is, typically, from 1 to 100,000 times the concentration of the active ingredient.

Additional ingredients that may be included in the formulations include but are not limited to tonicity enhancers, preservatives, solubilizers, non-toxic excipients, demulcents, sequestering agents, pH adjusting agents, co-solvents and viscosity building agents.

For the adjustment of the pH, preferably to a physiological pH, buffers may especially be useful. The pH of the present solutions should be maintained within the range of 4.0 to 8.0, more preferably about 4.0 to 6.0, more preferably about 6.5 to 7.8. Suitable buffers may be added, such as boric acid, sodium borate, potassium citrate, citric acid, sodium bicarbonate, TRIS, and various mixed phosphate buffers (including combinations of Na₂HPO₄, NaH₂PO₄ and KH₂PO₄) and mixtures thereof. Borate buffers are preferred. Generally, buffers will be used in amounts ranging from about 0.05 to 2.5 percent by weight, and preferably, from 0.1 to 1.5 percent.

Tonicity is adjusted, if needed, typically by tonicity enhancing agents. Such agents may, for example, be of ionic and/or non-ionic type. Examples of ionic tonicity enhancers are alkali metal or earth metal halides, such as, for example, CaCl₂, KBr, KCl, LiCl, NaI, NaBr or NaCl, Na₂SO₄ or boric acid. Non-ionic tonicity enhancing agents are, for example, urea, glycerol, sorbitol, mannitol, propylene glycol, or dextrose. The aqueous solutions of the present invention are typically adjusted with tonicity agents to approximate the osmotic pressure of normal lachrymal fluids which is equivalent to a 0.9% solution of sodium chloride or a 2.5% solution of glycerol. An osmolality of about 225 to 400 mOsm/kg is preferred, more preferably 280 to 320 mOsm.

In certain embodiments, the formulations additionally comprise a preservative. A preservative may typically be selected from a quaternary ammonium compound such as benzalkonium chloride, benzoxonium chloride or the like. Benzalkonium chloride is better described as: N-benzyl-N—(C8-C18 alkyl)-N, N-dimethylammonium chloride. Examples of preservatives different from quaternary ammonium salts are alkyl-mercury salts of thiosalicylic acid, such as, for example, thiomersal, phenylmercuric nitrate, phenylmercuric acetate or phenylmercuric borate, sodium perborate, sodium chlorite, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, sodium perborate, Germal® or sorbic acid. Preferred preservatives are quaternary ammonium compounds, in particular, benzalkonium chloride or its derivative such as Polyquad (see U.S. Pat. No. 4,407,791), alkyl-mercury salts and parabens. Where appropriate, a sufficient amount of preservative is added to the ophthalmic composition to ensure protection against secondary contaminations during use caused by bacteria and fungi.

In another embodiment, the ophthalmic formulations do not include a preservative since ocular preservatives and absorption enhancers can cause injury should be kept in mind (Kaur IP et al (2009) “Ocular preservatives: associated risks and newer options”, Cutan Ocul Toxicol 28:93-103 and Furrer P et al (2002) “Ocular tolerance of absorption enhancers in ophthalmic preparations”, Aaps Pharmsci 4:E2). Such formulations would be useful for patients who wear contact lenses, or those who use several topical ophthalmic drops and/or those with an already compromised ocular surface wherein limiting exposure to a preservative may be more desirable.

In a particular embodiment, said agent(s) may be incorporated, attached or carried on a contact lens using procedures known in the art. As an example of drug loading of drug-eluting contact lenses, as hydrogel contact lenses, a preliminary study for the treatment of glaucoma uses the beta-blocker timolol loaded into drug-eluting contact lenses, such as silicone-hydrogel contact lenses (see, for example, Jung H J et al. (2013) “Glaucoma therapy by extended release of timolol from nanoparticle loaded silicone-hydrogel contact lenses”, J Controlled Release 165:82-9). Of interest, in another preliminary study of drug-eluting contact lenses, two agents have been used for the purpose of treating glaucoma. (Hsu et al. (2015) “Dual drug delivery from vitamin E loaded contact lenses for glaucoma therapy”, Eur J Pharmaceut Biopharmaceut 94:312-21).

The formulations may additionally require the presence of a solubilizer, in particular if the active or the inactive ingredients tends to form a suspension or an emulsion. A solubilizer suitable for an above concerned composition, is for example, selected from the group consisting of tyloxapol, fatty acid glycerol polyethylene glycol esters, fatty acid polyethylene glycol esters, polyethylene glycols, glycerol ethers, a cyclodextrin (for example alpha-, beta- or gamma-cyclodextrin, e.g. alkylated, hydroxyalkylated, carboxyalkylated or alkyloxycarbonyl-alkylated derivatives, or mono- or diglycosyl-alpha-, beta- or gamma-cyclodextrin, mono- or dimaltosyl-alpha-, beta- or gamma-cyclodextrin or panosyl-cyclodextrin), polysorbate 20, polysorbate 80 or mixtures of those compounds. A specific example of a solubilizer is a reaction product of castor oil and ethylene oxide, for example the commercial products Cremophor EL® or Cremophor RH40®. Reaction products of castor oil and ethylene oxide have proved to be particularly good solubilizers that are tolerated extremely well by the eye. Another solubilizer is selected from tyloxapol and from a cyclodextrin. The concentration used depends especially on the concentration of the active ingredient. The amount added is typically sufficient to solubilize the active ingredient. For example, the concentration of the solubilizer is from 0.1 to 5000 times the concentration of the active ingredient.

The formulations may comprise further non-toxic excipients, such as, for example, emulsifiers, wetting agents or fillers, such as, for example, the polyethylene glycols designated 200, 300, 400 and 600, or Carbowax designated 1000, 1500, 4000, 6000 and 10000. The amount and type of excipient added is in accordance with the particular requirements and is generally in the range of from approximately 0.0001 to approximately 90% by weight.

Other compounds may also be added to the formulations of the present invention to increase the viscosity of the carrier. Examples of viscosity enhancing agents include, but are not limited to polysaccharides, such as hyaluronic acid and its salts, chondroitin sulfate and its salts, dextrans, various polymers of the cellulose family and vinyl polymers.

What is claimed is:

1. A method for preventing and/or treating one or more neurodegenerative disorders in a subject in need thereof comprising ophthalmically administering an amount of one or more agents effective to normalize neuronal autoregulation, mimic a physiological parasympathetic shift, modulate activities of the autonomic nervous system or any combination of the foregoing.

2. The method according to claim 1, wherein said neurodegenerative disorder results from neuronal autoregulation dysfunction.

3. The method according to claim 1, wherein said neurodegenerative disorder is an intraocular disorder.

4. The method according to claim 2, wherein said intraocular disorder is glaucoma, AMD diabetic retinopathy, retinitis pigmentosa, retinal vein occlusion, and retinopathy of prematurity.

5. The method according to claim 1 wherein said neurodegenerative disorder is a cerebral neurodegenerative disorder.

6. The method according to claim 5, wherein said cerebral neurodegenerative disorder is selected from the group consisting of Parkinson's disease, Alzheimer's disease, Lewy body dementia, Huntington's disease, or amyotrophic lateral sclerosis.

7. The method according to claim 1, wherein said subject is administered said agent in an amount at least about 5% below its lowest effective therapeutic ophthalmic dose.

8. The method according to claim 1, wherein said subject is administered a parasympathomimetic agent and/or an anti-sympathetic agent.

9. The method according to claim 8, wherein said subject is administered said parasympathomimetic agent and/or anti-sympathetic agent in an amount at least about 10% below its lowest effective therapeutic ophthalmic dose.

10. The method according to claim 8, wherein said anti-sympathetic agent is an alpha agonist, beta-blocker or angiotensin blocker.

11. The method according to claim 8, wherein said anti-sympathetic agent is a beta-blocker, wherein said beta-blocker is selected from the group consisting of carvedilol, nebivolol, bexolol, propanolol.

12. The method according to claim 8, wherein said parasympathomimetic agent is selected from the group consisting of pilocarpine, ecothiopate, demecarium bromide, diisopropyl fluorophosphate and carbachol.

13. The method according to claim 8, wherein said anti-sympathetic agent is an angiotensin blocker, wherein said angiotensin blocker is an ACE inhibitor or an angiotensin II receptor antagonist.

14. The method according to claim 8, wherein said parasympathomimetic agent and ant-sympathetic agent are administered in the form of a composition.

15. The method according to claim 1, wherein said agent is ophthalmically administered by administering said agent to the eye topically, by intraocular injection or by intravitreal injection.

16. The method according to claim 1 wherein said effect of said agent on neuronal autoregulation is determined by measuring at least one of (a) glutamate level; (b) inflammation level in the eye and/or brain; (c) amyloid level; (d) vasodilation activity; (e) acetylcholine level in said subject.

17. The method according to claim 1, wherein said physiological parasympathetic shift is determined by measuring the level of chronic sympathetic activation in said subject.

18. The method according to claim 1, wherein the modulating activity of the autonomic nervous system is determined by imaging.

19. The method according to claim 1, wherein said physiological parasympathetic shift is mimicked via parasympathetic activation.
